(19) United States
(12) Patent Application Publication
(10) Pub. No.: US 2018/0264476 A1
(43) Pub. Date: Sep. 20, 2018

(54) APPARATUS, SYSTEMS AND METHODS FOR DYNAMIC FLUX AMPLIFICATION OF SAMPLES

(71) Applicant: Fluoresentric, Inc., Park City, UT (US)

(72) Inventors: Brian CAPLIN, Park City, UT (US); Brian HICKE, Park City, UT (US); Bryson GREEN, Herber City, UT (US)

(21) Appl. No.: 15/760,360

(22) PCT Filed: Sep. 16, 2016
(86) PCT No.: PCT/US2016/052335

§ 371 Date: Mar. 15, 2018

Related U.S. Application Data
(60) Provisional application No. 62/219,552, filed on Sep. 16, 2015.

(51) Int. Cl. B01L 7/00; C12Q 1/686

(52) U.S. Cl. CPC B01L 7/52; C12Q 1/686; B01L 2300/0854; B01L 2300/025; B01L 2300/1822; B01L 2300/0663; B01L 2300/1805

(57) ABSTRACT

Embodiments of the current disclosure are directed towards apparatus, methods and systems configured for dynamic flux amplification of samples in reaction vessels. In some embodiments, an apparatus comprising a reaction vessel and a heat source is disclosed. The reaction vessel may include a first wall and an opposing second wall positioned so as to define a sample chamber therebetween, a width of the sample chamber being less than about 2 mm. The heat source may be configured to vary a first temperature of the first wall and a second temperature of the second wall such that a temperature difference between the first temperature and the second temperature induces thermal cycling in a solution contained within the sample chamber of the reaction vessel.

### CROSS REFERENCE TO RELATED APPLICATIONS

This application claims priority to U.S. Provisional Patent Application No. 62/219,552, filed Sep. 16, 2015, titled “DYNAMIC FLUX AMPLIFICATION INSTRUMENT”, the disclosure of which is incorporated by reference herein in its entirety.

### BACKGROUND

Molecular diagnostic methods have become increasingly important diagnostic tools since the methods were first developed over 30 years ago. With the current state of sample amplification methods and instruments, the time it takes to obtain a result can be about an hour or even more. Further, conventional thermal cycling instruments are generally too expensive to be practical for average clinical settings, and too bulky for field use.

### SUMMARY

Embodiments of the present disclosure are directed towards apparatus, methods and systems configured for dynamic flux amplification of samples in reaction vessels. In some embodiments, an apparatus comprising a reaction vessel and a heat source is disclosed. The reaction vessel may include a first wall and an opposing second wall positioned so as to define a sample chamber therebetween, a width of the sample chamber being less than about 2 mm. The heat source may be configured to vary a first temperature of the first wall and a second temperature of the second wall such that a temperature difference between the first temperature and the second temperature induces thermal cycling in a solution contained within the sample chamber of the reaction vessel. In some embodiments, the maximum width of the sample chamber can be in a range from about 0.5 mm to about 1.0 mm. In some embodiments, the reaction vessel may be a closed system.

In some embodiments, the heat source includes a first thermal transfer block in thermal communication with the first wall and a second thermal transfer block in thermal communication with the second wall. Further, the heat source may include a first plate in direct thermal contact with a side of the first wall, and a second plate in direct thermal contact with a side of the second wall. In addition, the heat source may include a first Peltier device for varying the temperature of the first wall and a second Peltier device for varying the temperature of the second wall. In some embodiment, the above-noted temperature difference may be in a range from about 10° C. to about 40° C., or in a range from about 40° C. to about 75° C.

In some embodiments, a system comprising an apparatus, a processor, a light source and a detector is disclosed. The apparatus may include a reaction vessel including a first wall and an opposing second wall positioned so as to define a sample chamber therebetween, a maximum width of the sample chamber being less than about 2 mm; and a heat source including a first thermal transfer block and/or a second thermal transfer block, the first thermal transfer block in thermal communication with the first wall and configured to vary a first temperature of the first wall, the second thermal transfer block in thermal communication with the second wall and configured to vary a second temperature of the second wall. Further, the apparatus may include a processor, operatively coupled to the heat source, and configured to determine a characteristic of a current for flowing through: the first thermal transfer block so as to establish the first temperature of the first wall, and/or the second thermal transfer block so as to establish the second temperature of the second wall. In addition, the apparatus may include a light source configured for irradiating the solution in the sample chamber of the reaction vessel; and a detector, operatively coupled to the reaction vessel, the detector configured for detecting fluorescence emitted by the solution in the sample chamber. In some embodiments, the maximum width of the sample chamber ranges from about 0.5 mm to about 1.0 mm.

Further, the system may comprise a conduit for transmitting the fluorescence light exiting the sample chamber to the detector. In addition, the system may comprise a display, operatively coupled to the detector, and configured to display a graphical representation of the fluorescence from the sample chamber. In some embodiments, the characteristic of the current includes an amount and/or direction of the current flowing through the first thermal transfer block and/or the second thermal transfer block. In addition, the characteristic of the current includes a duration of time for flowing the current through the first thermal transfer block and/or the second thermal transfer block. In some embodiments, the difference of the first temperature and the second temperature ranges from about 10° C. to about 40° C. In some embodiments, the detector includes a smartphone.

Some embodiments of the current disclosure also include a method, comprising the steps of: forming a reaction vessel including a first wall and an opposing second wall positioned so as to define a sample chamber therebetween, a width of the sample chamber being less than about 2 mm; and coupling a heat source to the first wall and the second wall, the heat source configured to vary a first temperature of the first wall and a second temperature of the second wall such that during use, a temperature difference between the first temperature and the second temperature induces thermal cycling in a solution contained within the sample chamber of the reaction vessel.

In addition, some embodiments of the current disclosure also include a method comprising the steps of establishing, via a heat source, a temperature difference between a first wall and an opposing second wall of a reaction vessel, the first wall and the second wall arranged in close proximity to each other so as to define a sample chamber therebetween, the sample chamber having a width of less than about 2 mm; irradiating the sample chamber with light, the sample chamber having a solution disposed therein; and detecting, via a detector operatively coupled to the reaction vessel, fluorescence emitted by the solution, wherein the temperature difference between the first wall and the second wall is configured to induce thermal cycling of the solution in the sample chamber. In some embodiments, establishing a temperature difference between the first wall and the second wall includes varying a first temperature of the first wall and/or a second temperature of the second wall by flowing a current through a first thermal transfer block and/or a second thermal transfer block, the first thermal transfer block and/or the second thermal transfer block being in thermal communication with the first wall and/or the second wall, respectively. Further, the method includes the step of determining, via a processor operatively coupled to the first thermal transfer block and/or the second thermal transfer block, an amount, a direction and/or a duration of the current to flow through the first thermal transfer block and/or the second thermal transfer block to maintain the first temperature and/or the second temperature, respectively. In addition, the method also includes the step of displaying, at a display of the detector and/or an external display operatively coupled to the detector, a graphical representation of the fluorescence light emitted by the content of the sample chamber.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 shows an example block diagram of a system for dynamically amplifying samples via convective thermal cycling, according to some embodiments.

FIG. 2 shows an example illustration of a thermo-cycling device coupled to a light source configured for illuminating the reaction vessel of the thermo-cycling device, according to some embodiments.

FIG. 3 shows an example illustration of a side view of the thermo-cycling device including a reaction vessel in direct contact with thermal transfer blocks, according to some embodiments.

FIG. 4 shows an example illustration of the thermo-cycling of a sample within a reaction vessel, according to some embodiments.

FIG. 5 shows an example illustration of a thermo-cycling device coupled to a light source configured for illuminating the reaction vessel of the thermo-cycling device and a fluorescence detector configured for detecting fluorescence emitted by sample contained within the reaction vessel, according to some embodiments.

FIG. 6 shows another example embodiment of FIG. 5 where the detector is a smartphone.

FIG. 7 shows an example comparative plot illustrating the time it takes to obtain results when using the devices disclosed in the instant disclosure versus standard polymerase chain reaction (PCR) devices, according to some embodiments.

FIG. 8 shows an example combined electronic/mechanical diagram illustrating the use of feedback-based proportional-integral-derivative (PID) controller to control temperatures of thermal transfer blocks, according to some embodiments.

FIG. 9 shows an example graphical representation of a design for overlapping primer annealing temperatures and template denaturation temperatures, according to some embodiments.

FIG. 10 provides an example illustration of conventional amplification products by real time PCR, according to some embodiments.

FIG. 11 shows an example graph showing high temperature PCR amplification of the same template used in FIG. 10, according to some embodiments.

### DETAILED DESCRIPTION OF SOME OF THE EMBODIMENTS

Standard polymerase chain reaction (PCR) devices and methods of using thereof have been important tools of molecular diagnostics technologies. These standard tools may be used to obtain accurate, informative and rapid results when used to study a variety of samples, for example, when used for diagnostic or environmental detections of particles or chemicals in samples (e.g., detecting the presence of viruses in blood samples, and/or the like). In some embodiments, however, one may wish to reduce the length of time it can take to obtain the results when using these tools, or to speed up the thermal cycling processes that are employed when using these tools to activate the chemical reactions (such as PCR). For example, thermal cycling in PCR applications currently can take hours, which can be an undesirably long time at least for some applications, such as for point-of-care field performances of molecular diagnosis of infectious diseases in humans and animals where one may wish to reduce the time duration to mere minutes. Further, one may also desire to reduce the size and/or weight of the devices so as to facilitate and increase the use of these devices in locations that may be difficult to access (e.g., field performances of diagnosis and/or detections in remote areas where standard PCR equipment may not be readily available). In some embodiments, devices may be built to be portable so as to allow the deployment of the devices for field performances at point of care locations where there may be pronounced needs for the devices but not ready or adequate access.

In some embodiments, one may manage various types of chemical reactions in a sample by controlling the temperature of the sample and/or its environs. For example, one may enhance the amplification of a segment of nucleic acid in a DNA synthesis reaction by thermo-cycling the reaction components, including the nucleic acid segment, between elevated and reduced temperatures so as to exponentially amplify the segment via the processes of dehybridization, annealing, and polymerization.

The terms “thermal cycling”, “thermo-cycling”, and variants thereof, as used herein can be understood to mean at least one change of temperature, i.e. increase or decrease of temperature, in the environment to which the reaction components are exposed. For example, one may thermally cycle a sample by shifting the temperature of the sample (e.g., by an application of a heater/cooler to the walls of the container holding the sample) from one value to another that is higher or lower, and then returning the temperature back to the initial value (or cycle the temperature between higher and lower values without necessarily repeating the temperature values). In some embodiments of the present disclosure, different temperature values may be applied to different sections of the container holding the sample (e.g., a higher temperature to one wall of the container and a lower one to an opposing wall), and the sample may be allowed to cycle through the container, effectively the sample thermally cycles within the container. Such types of thermal or temperature cycling may be performed once or repeated as many times as required by the particular chemical reaction.

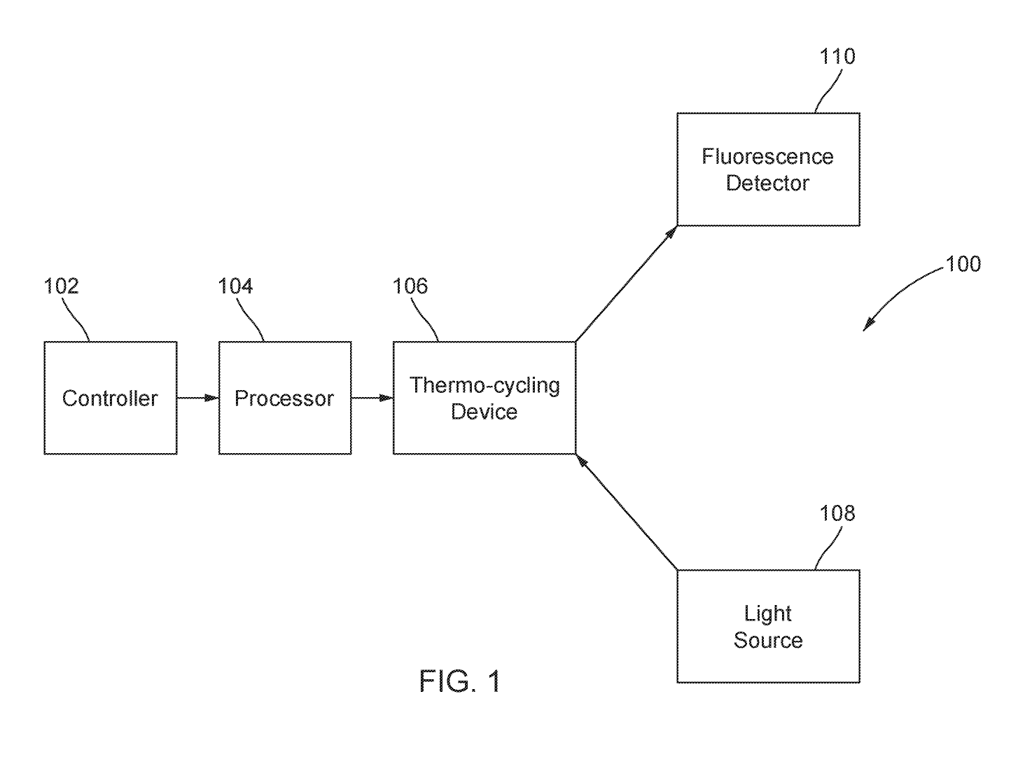

In some embodiments, the present disclosure presents a system for use in molecular diagnostic/detection technologies that can be utilized to dynamically amplify samples via convective thermal cycling. With reference to FIG.1, in some embodiments, the system 100 includes a thermo-cycling device 106 configured to thermally cycle samples contained within a reaction vessel (not shown in FIG. 1) of the device 106. For example, the thermo-cycling device 106 can include one or more heat sources, and the reaction vessel can be coupled to the heat source(s) that is configured to control and/or vary the temperatures of the walls of the reaction vessel. Differences in the temperatures of walls (e.g., opposing walls of the reaction vessel) may then facilitate, result in, and/or otherwise induce the thermal cycling of the sample contained within the reaction vessel. In some embodiments, the system 100 may also include a controller 102 that is configured to determine temperature levels that are conducive to enhanced operation of the thermal cycling device 106. For example, the controller 102 may determine desired temperatures for a thermal cycling operation, and then communicate the determined temperatures to a processor 104 operatively coupled to the thermo-cycling device 106. In some embodiments, for example when the heat sources include thermal transfer blocks in thermal communication with the reaction vessel (or walls thereof), the processor 104 can determine the characteristics of an electric current that may be flown into the thermal transfer blocks so as to generate the desired temperatures in the walls of the reaction vessel.

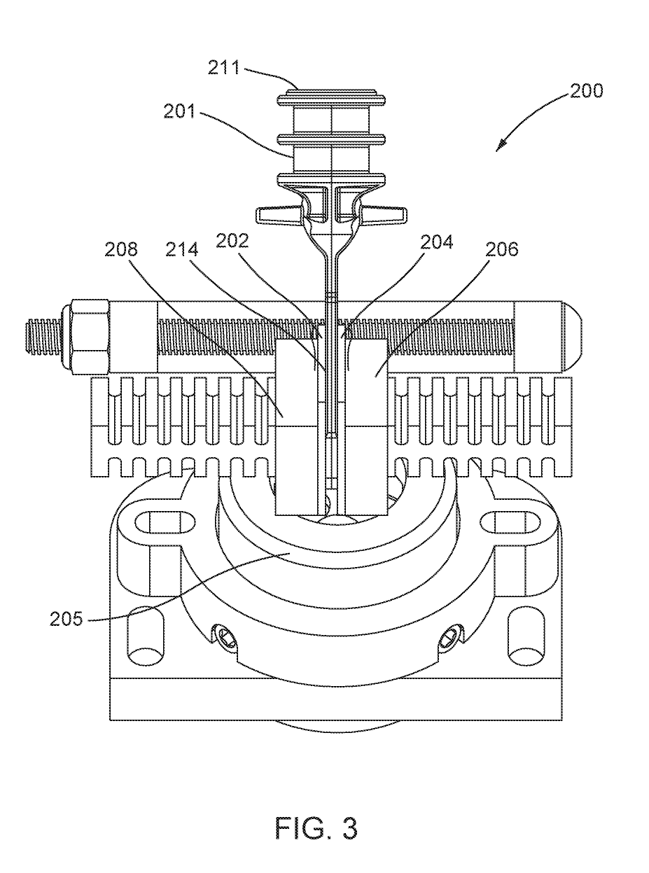

In some embodiments, the system 100 may also include a light source 108 for illuminating or irradiating the samples contained within so as to interrogate the samples before, during and/or after thermal cycling. In response to the illumination, in some embodiments, the samples may emit a fluorescence light, which can then be detected by a fluorescence detector 110 that may be operatively coupled to the thermo-cycling device 106. FIG. 3 provides an example illustration of a thermo-cycling device 200 coupled to a light source 206, which is configured for illuminating the reaction vessel of the thermo-cycling device 206. In some embodiments, the light source 108 and the fluorescence detector 110 may be in communication with the controller 102 while being coupled to the thermo-cycling device 106. In some embodiments, the light source 108 and the fluorescence detector 110 may not be in communication with the thermo-cycling device 106, but may instead be in communication with the controller 102. In some embodiments, the operation of the light source 108 may be separate and independent from the operation of the fluorescence detector 110 (i.e., the optical detectors), i.e., the light source 108 and the fluorescence detector 110 may not be operated simultaneously. In some embodiments, however, both the light source 108 and the optical or fluorescence detector 110 may be operated simultaneously.

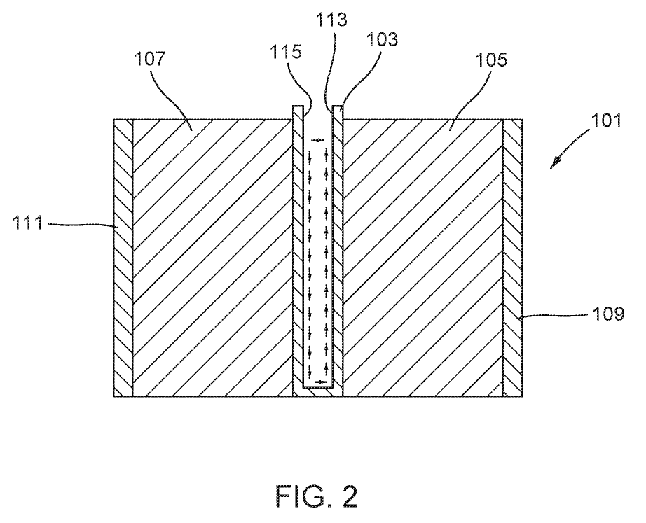

In some embodiments, the thermo-cycling of a sample within a container or a vessel may be accomplished by shifting the temperature of the sample or the container between higher and lower values. In other embodiments, different sections of the container may be maintained at different temperature values and the sample within the container may be allowed to circulate within the container, thereby effecting the thermal cycling of the sample. For example, with reference to FIG. 2, in some embodiments, a reaction vessel 103 containing a sample in its sample chamber may be in thermal communication (e.g., direct contact) with at least two thermal transfer blocks 105 and 107, which are configured to maintain the temperature of the respective reaction vessel walls 113, 115 to which they are contacted with at different temperatures from each other. Each thermal transfer block 105, 107 may in turn be in thermal communication with temperature regulators 109, 111 such as Peltier devices that are configured to heat or cool the thermal transfer blocks, which in turn heat or cool the walls 113, 115 of the reaction vessel 103. In some embodiments, the thermal transfer blocks 105 and 107 may include the Peltier devices 109 and 111 (e.g., as an integral component) or may be coupled to the devices. In some embodiments, there may be additional thermal blocks (not shown) sandwiching the reaction vessel 103, the thermal transfer blocks 105, 107, and the temperature regulators 109, 111 so as to maintain the desired temperature distribution within the reaction vessel 103. In some embodiments, there may be a plurality of thermal transfer blocks and/or Peltier devices on each side of the reaction vessel walls 113, 115. For example, each vessel wall 113, 115 may be in thermal communication (directly or otherwise) with one, two, three, and/or the like, number of thermal transfer blocks and/or Peltier devices.

In some embodiments, a controller (such as the controller 102 in FIG. 1) may determine or receive as an input the temperature values for the two opposing walls 113, 115 of the reaction vessel 103 so as to facilitate the thermal cycling of a sample contained within the sample chamber of the reaction vessel 103. For example, when thermo-cycling DNA segments, a higher temperature of about 95° C. and a lower temperature in the range of 40° C.-60° C. (e.g., for annealing) or 70° C.-75° C. (e.g., for polymerization) may be selected (by the controller and/or as input) for applying to the walls of the reaction vessel 103. In some embodiments, for general PCR or other applications, the disclosed system or device of the instant disclosure may be configured to maintain a desired temperature difference between the two opposing walls 113, 115 of the reaction vessel 103. For example, a temperature difference in the range from about 0° C. to about 99° C. can be maintained between the walls 113, 115. In some embodiments, the temperature difference can be in the range from about 5° C. to about 60° C., from about 10° C. to about 50° C., from about 10° C. to about 40° C., from about 10° C. to about 30° C., from about 10° C. to about 20° C., from about 10° C. to about 15° C., from about 20° C. to about 50° C., from about 25° C. to about 40° C., from about 30° C. to about 40° C., including values and subranges therebetween. Such temperature differences can be established by setting the temperature of one of the walls (one of 113 and 115) of the reaction vessel 103 at a given temperature and raising or lowering the temperature of the opposing wall (the other of 113 and 115) by an amount of the desired temperature difference. In some embodiments, the walls 113, 115 may be configured to maintain these established temperatures within a small range, for example, 3° C., about 2° C., about 1° C., about 0.5° C., about 0.1° C., and/or the like, of the established temperatures.

In some embodiments, a temperature gradient may occur across the width of the reaction vessel 103 from the higher temperature wall to the lower temperature wall. However, thermal losses due to mass of the wall and mass of the thermal transfer blocks may be minimal.

In some embodiments, upon determining or receiving as an input the temperature difference between the opposing walls 113, 115, and/or the temperature for each wall 113, 115, the controller may determine the characteristics of current that may be flown through/provided to each thermal transfer block (which may include the Peltier devices) to set the temperature difference between the walls 113, 115 at about the determined or input temperature difference (or equivalently, set the temperatures of the opposing walls 113, 115 at the determined or input values). Examples of such current characteristics include the amount of the current, the direction of the current, the time duration that the temperature values should be maintained (e.g., the time duration the current should be flowing), and/or the like.

For example, for a given sample contained within the sample chamber of a reaction vessel 103, in some embodiments, a user may determine that a temperature difference in the range of from about 10° C. to about 20° C. may result in optimal thermal cycling of the sample. In some embodiments, these values may be provided to a controller (102 in FIG. 1) by a user via a user input interface operationally coupled to the device 101. In such embodiments, the controller 102 may have a degree of freedom to choose the temperatures for each thermal transfer block (105 or 107) or wall (113 or 115) of the reaction vessel that would result in the temperature difference (i.e., for a given temperature difference, there may be several pairs of temperature for the opposing walls 113, 115 or thermal transfer blocks 105, 107 that result in the same temperature difference). In such embodiments, the controller may determine the temperature for each thermal transfer block 105, 107 or vessel wall 113, 115 based on factors such as the type, amount, and/or the like, of sample contained within the reaction vessel 103, the desired rate of thermo-cycling, and/or the like. The controller may then calculate the current characteristics such as amount, direction, duration, and/or the like, of the current to be flown through each thermal transfer block 105, 107 so as to realize the desired temperature difference between the walls 113, 115 of the reaction vessel 103.

Upon determining the characteristics of the current flows, in some embodiments, the controller 102 may transmit a signal with instructions to a processor (the processor 104 in FIG. 1, for example) operably coupled to the thermo-cycling device 101 to generate and send the currents to the thermal transfer blocks 105, 107. Based on the instructions, the processor 104 may then activate the thermal transfer blocks 105, 107 (e.g., the Peltier devices of the thermal transfer blocks) to drive the currents through the thermal transfer blocks 105, 107 to set the temperatures of the thermal transfer blocks 105, 107 and/or the reaction vessel walls to establish the desired temperature difference between the vessel walls 113, 115. For example, one of the walls (one of 113 and 115) of the reaction vessel 103 may have a higher temperature while the opposing wall (the other of 113 and 115) may have a lower temperature. The difference in temperature between the two walls 113, 115 of the reaction vessel 103 may then create a temperature gradient within the sample chamber of the reaction vessel 103, which can lead to the portion of the sample that is proximal to the higher temperature wall being at a higher temperature than the portion of the sample that is proximal to the lower temperature wall. In some embodiments, the colder portion of the sample can then sink within the sample chamber while the hotter portion rises, setting up a convective current of the sample in the sample chamber within the reaction vessel 103, as shown in FIG. 2. During the circulation of the convective current, in some embodiments, the entire sample is effectively being exposed to higher and lower temperatures in an alternative manner, i.e., the entire sample within the reaction vessel may become thermally cycled, provided the temperature differential (i.e., temperature difference between the vessel walls 113, 115) is maintained.

With reference to FIG. 3, in some embodiments, the thermo-cycling device 200 includes an opening 211 that is configured to allow the introduction of samples and/or other ingredients into the sample chamber of the reaction vessel 214. For example, a channel 201 may connect the opening 211 to the sample chamber. In some embodiments, the sample chamber of the reaction vessel 214 may be defined by the inner surfaces of two opposing walls (113 and 115 in FIG. 2) of the reaction vessel 214 that may be in close proximity to each other. In general, the reaction vessel 214 may be shaped and sized in a manner that facilitates the thermo-cycling of the samples contained within. For example, the opposing inside surfaces of the walls 113, 115 of the reaction vessel 214 may be regularly shaped (e.g., flat and rectangular) such that the width of the sample chamber (i.e., the separation distance between the inner surfaces of the reaction vessel walls 113, 115) may be uniform throughout the chamber.

In some embodiments, there may be variations in the temperatures of one or both of the walls before, during and/or after a partial or complete convective amplification reaction. For example, the temperature variations may be such that the device may be in a cold hold where it is incubating a low temperature reaction (e.g., reverse transcription reaction). Another example may be the temperature variations are such that the device is essentially ‘refrigerating’ the sample. In some embodiments, on the other end of the amplification, the blocks may be uniformly heated to generate a melt profile of the amplified sample. In some embodiments, e.g. during the amplification process, there may be a transition from one reaction to the next in a XCRgen2 multiplex method.

In some embodiments, the width of the sample chamber may not be uniform within the reaction vessel, and in such embodiments, a linear quantity characterizing the effective separation of the inner surfaces of the walls may be used to represent the separation distance (i.e., the “width” of the sample chamber). For example, the average separation distance between the inner surfaces of the walls 113, 115 of the reaction vessel may be used to define the “width” of the sample chamber (e.g., a mathematical quantity averaged over the inner surfaces of the reaction vessel walls 113, 115). In some embodiments, a single value such as the closest separation distance between the opposing inner surfaces may define the width of the sample chamber. In any of the above noted embodiments, the width of the sample chamber can be in the range from about 0.5 mm to about 2 mm, from about 1 mm to about 2 mm, from about 1 mm to about 1.5 mm, from about 0.5 mm to about 1.5 mm, from about 0.5 mm to about 1 mm, from about 1 mm to about 1.8 mm, from about 1.2 mm to about 1.6 mm, from about 1.4 mm to about 1.6 mm, including values and subranges therebetween.

In some embodiments, the outside surfaces of the opposing walls 113, 115 of the reaction vessel 214 may be shaped and sized so as to form direct contact with thermal plates 202, 204 (e.g., the surfaces may be flat). The contact facilitates thermal communication between thermal transfer blocks 206, 208 and the surfaces of the walls 113, 115 of the reaction vessel 214. In some embodiments, the thermal transfer blocks 206, 208 may include the plates 202, 204, respectively, i.e., the plates 202, 204 may be integral or monolithic components of the thermal transfer blocks 206, 208, respectively. In some embodiments, the plates 202, 204 may be separate from, but instead be in thermal communication with, the thermal transfer blocks 206, 208, respectively.

In some embodiments, the thermal transfer blocks 206, 208 can generate heat for increasing the temperature of the plates 202, 204 and/or reaction reservoir walls. In some embodiments, the thermal transfer blocks 206, 208 may also be configured for lowering the temperature of the plates 202, 204 and/or reaction reservoir walls. For example, the thermal transfer blocks 206, 208 may include Peltier devices capable of heating or cooling objects the blocks or devices are in thermal communication with. Said another way, the thermal transfer blocks can control (e.g., raise, lower, keep substantially constant, and/or the like) the temperatures of the walls of the reaction vessel 214 and/or the plates to which the walls are in thermal communication with. In some embodiments, the thermal transfer blocks 206, 208 may include any types of temperature regulators that are capable raising and/or lowering temperatures, including, for example, heat pumps, refrigerators, thermoelectric coolers, heaters, and/or the like. In some embodiments, the thermal transfer blocks are configured to have temperatures in the range from about 0° C. to about 99° C., from about 0° C. to about 80° C., from about 0° C. to about 60° C., from about 0° C. to about 40° C., from about 0° C. to about 20° C., from about 20° C. to about 99° C., from about 40° C. to about 99° C., from about 60° C. to about 99° C., from about 80° C. to about 99° C., including values and subranges therebetween. In some embodiments, the rate of temperature change of the thermal transfer blocks may be configured so as to allow a substantially precise control of the heating process. For example, the rate of temperature change in the thermal transfer blocks may be in the range from about 0.01° C./sec to about 5° C./sec, from about 0.05° C./sec to about 5° C./sec, from about 0.1° C./sec to about 0.3° C./sec, from about 0.1° C./sec to about 0.1° C./sec, about 0.5° C./sec, including values and subranges therebetween.

In some embodiments, the spacing between the thermal transfer blocks 206, 208 (or the plates 202, 204 such as when the plates are separate or distinct from the thermal transfer blocks 206, 208) may be configured to allow the placement or positioning of the reaction vessel 204 in between the thermal transfer blocks 206 and 208 or in between the plates 202 and 204. In some embodiments, this spacing may be kept low for a variety of reasons, including minimizing the size of the thermo-cycling device 200, enhancing thermo-cycling within the reaction vessel 214 by maintaining desired temperature gradient within the vessel 214, and/or the like. For example, the spacing may be in the range from about 0.05 mm to about 0.1 mm, about 0.1 mm to about 1 mm, about 1 mm to about 3 mm, from about 1 mm to about 2 mm, from about 1 mm to about 1.6 mm, from about 1.4 mm to about 1.6 mm, including values and subranges therebetween.

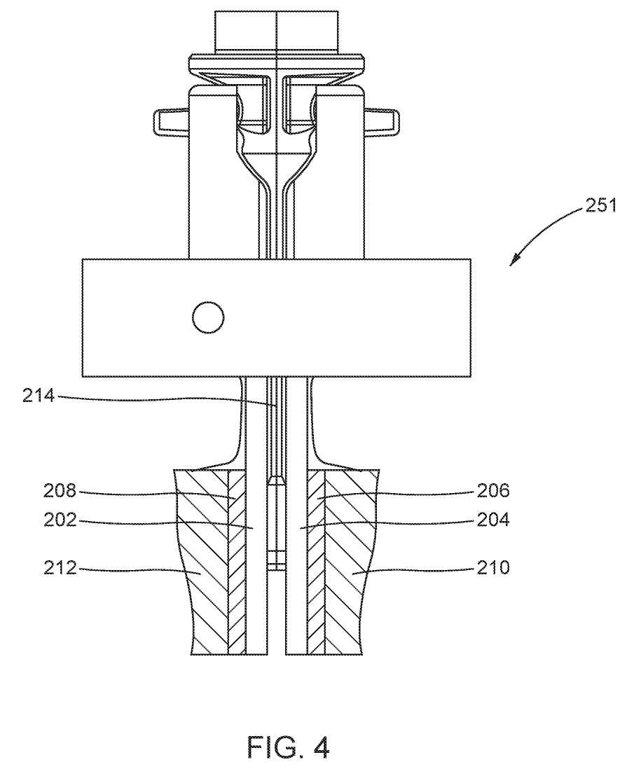

FIG. 4 shows a close-up side view of the thermo-cycling device 251 including the reaction vessel in thermal communication with heat sources, which may include thermal transfer blocks, thermal plates and thermal blocks, according to some embodiments. For example, a pair of thermal plates 202 and 204 may be separated from each other by a separation distance configured to allow the insertion of a reaction vessel 214 in between the thermal plates 202 and 204. Each of these thermal plates 202, 204 may in turn be in thermal communication with a temperature regulator 206, 208 such as a Peltier device. The thermal communication between the temperature regulators 206, 208 and the respective thermal plates 202, 204 may allow for the temperatures of the thermal plates 202, 204 to be varied, which in turn can vary the temperatures of the walls of the reaction vessel 214. In some embodiments, the heat source of a thermo-cycling device 251 can include a thermal block 210, 212 configured to maintain the desired temperatures of the one or more of the thermal plates 202, 204 and the walls of the reaction vessel 214. For example, the thermal blocks 202, 204 can be used to insulate the various components of the thermo-cycling device 251, which may include the reaction vessel 214, the temperature regulators 206, 208 (such as Peltier devices), the thermal plates 202, 204, and/or the like. In some embodiments, the proper maintenance of the temperatures of the reaction vessel walls (and as such, the vessel itself and contents contained within) may then facilitate an efficient thermo-cycling of the contents, leading to a rapid, power efficient operation of the system/devices for molecular diagnosis/detection.

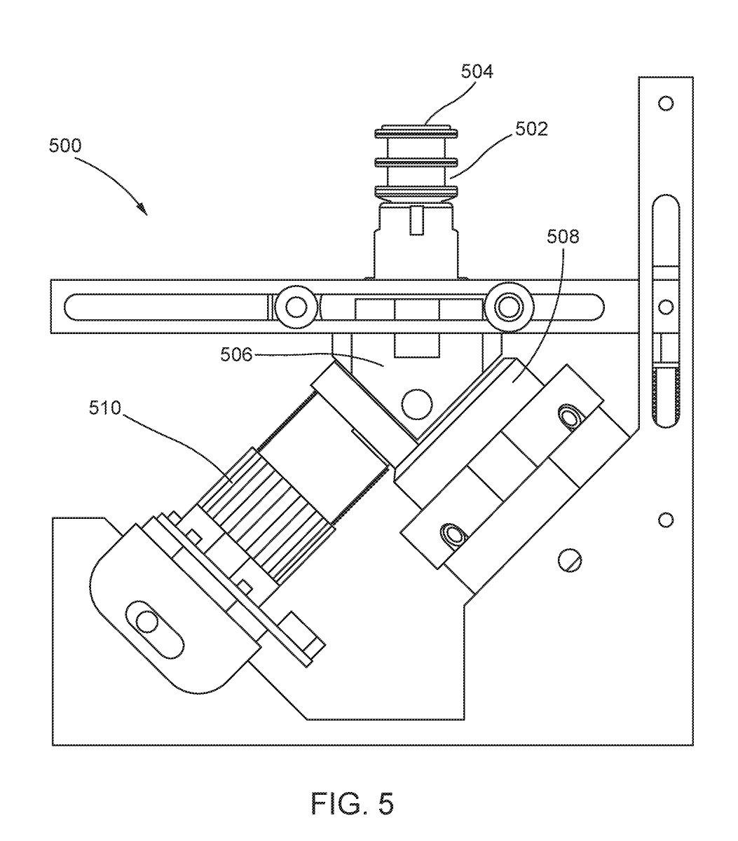

In some embodiments, the thermal cycling of the sample within the sample chamber of the reaction vessel may facilitate the production of reaction products, which one can then detect using a variety of detection mechanisms such as but not limited to optical interrogation (e.g., using fluorescence techniques, spectroscopic techniques, and/or the like). In some embodiments, the reaction process itself may be monitored using these detection techniques. For example, with reference to FIG. 5, in some embodiments, a molecular diagnostic/detection set-up 500 including components for optically detecting or monitoring samples contained within a sample chamber of a reaction vessel 506 is shown. The components may include one or more light sources 508 and one or more optical detectors 510 may be operatively coupled to a reaction vessel 506 so as to illuminate the samples with light and trigger the emission of florescence light by the reaction products and other components of the reaction. Some walls of the reaction vessel 506 (e.g., walls that are different from the walls in thermal communication with the thermal transfer blocks) may have ports that serve as entry ports for allowing light in from the light sources 508 and exit ports for allowing fluorescence light emitted by the sample to leave the sample chamber. In some embodiments, the light from the light sources 508 may be guided into the reaction vessel 506 via light guiding components such as optical fibers and/or other optics (lenses, filters, and/or the like). For example, in some embodiments, the light sources 508 may be associated with optical filters configured for selecting different components (e.g., wavelengths) of the light prior to radiating the reaction vessel and its contents. For example, an optical filter (not shown) may be positioned on the light path of each one of the one or more lights sources 508, and the filters may allow only certain wavelengths to pass and illuminate the sample chamber.

In some embodiments, the optical detector 510 may include one or more cameras configured to receive fluorescence light released by the sample in the sample chamber of the reaction vessel 506. Similarly, the optical detector 510 may be associated with optical filters that are configured to allow only certain wavelength components of the emitted fluorescence light to pass and reach the optical detector 510. For example, each optical reader 510 may have an N-Band optical filter positioned so as to filter the fluorescence entering the optical detectors 510 or cameras.

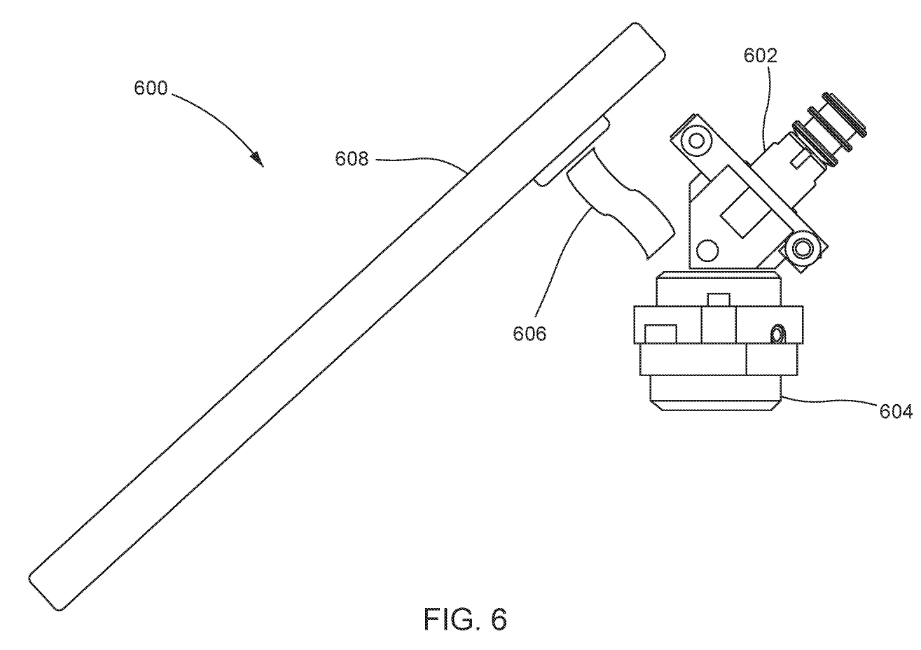

In some embodiments, the disclosed device or system may not include a built-in (e.g., integral) detection system, but rather may be operatively coupled to an external detector. This may be in particular the case when one wishes to reduce the size and weight of the molecular diagnosis or detection device (e.g., to facilitate the portability of the device/system). With reference to FIG. 6, in such embodiments, a smartphone 608 with camera functionality may be used in place of, or in addition to, an optical detector. In some embodiments, a light guiding system such as an optical waveguide 406 may be used to direct the emitted fluorescence light to the smartphone's camera input port. In some embodiments, the optical detector 510 of FIG. 5 and/or the smartphone 608 of FIG. 6 may be equipped with a mechanism to display information related to the received fluorescence light on the optical detector 510 and/or the smartphone 608, respectively. For example, the optical detector and/or the smartphone may include a display mechanism that is configured to present some representation of the level of the fluorescence light.

In some embodiments, spectroscopic techniques can also be used for monitoring and/or detecting samples instead of, or in conjunction with, the optical detection techniques disclosed herein.

### EXAMPLE 1

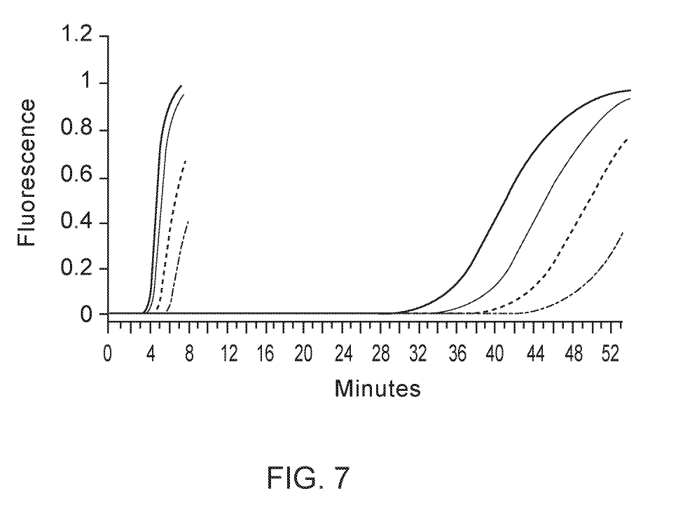

With reference to FIG. 7, in some embodiments, an example experimental plot illustrating the efficiency of the disclosed device/system is depicted. The plot shows an almost eight to ten-fold decrease in the time it takes to obtain results when using the diagnosis/detection device/system disclosed herein versus standard PCR systems. The plot compares the time it took to perform and obtain fluorescence results for an Xtreme Chain Reaction (XCR™) assaying process versus PCR for different quantities (702 and 710 for about 14000 copies, 704 and 712 for about 1400 copies, 706 and 714 for about 140 copies, 708 and 716 for about 14 copies) of human genomic DNA samples. Details of XCR amplification methods and systems have been presented in U.S. Pat. No. 9,353,408, filed Aug. 13, 2015, entitled “Dynamic Flux Nucleic Acid Sequence Amplification,” and U.S. Pat. No. 9,139,882, filed Nov. 22, 2010, entitled “System and Method for High Resolution Analysis of Nucleic Acids to Detect Sequence Variations,” incorporated herein by reference in their entireties. Comparing the results for the XCR™ device versus standard PCR devices, in some embodiments, the fluorescence results of the former are obtained eight to ten times earlier, and further that, the process of reaction products formation once thermo-cycling initiates is much faster in the case of the XCR™ results. In other words, the disclosed device/system employing XCR™ methodology allows for reactions products to start forming much earlier (by a factor of about 8 to 10) and faster (by factor or about 2 to 3 (corresponding to slopes of the curves 702, 704, 706 and 708 versus 710, 712, 714 and 716)).

In some embodiments, the power consumption of the apparatus, methods and systems disclosed herein can be much lower than that of a standard PCR application, thereby enhancing the portability and longevity of the disclosed device in use. For example, as shown in FIG. 7, there may be up to a factor of 10 reduction in the time duration it takes to obtain diagnosis and/or detection results when using the disclosed device, which may be associated with a reduction in the amount of power that may be consumed by the device during operation. Other sources of power consumption reductions can be the use of modern power efficient electronics with reduced operation voltages in the components of the thermo-cycling system.

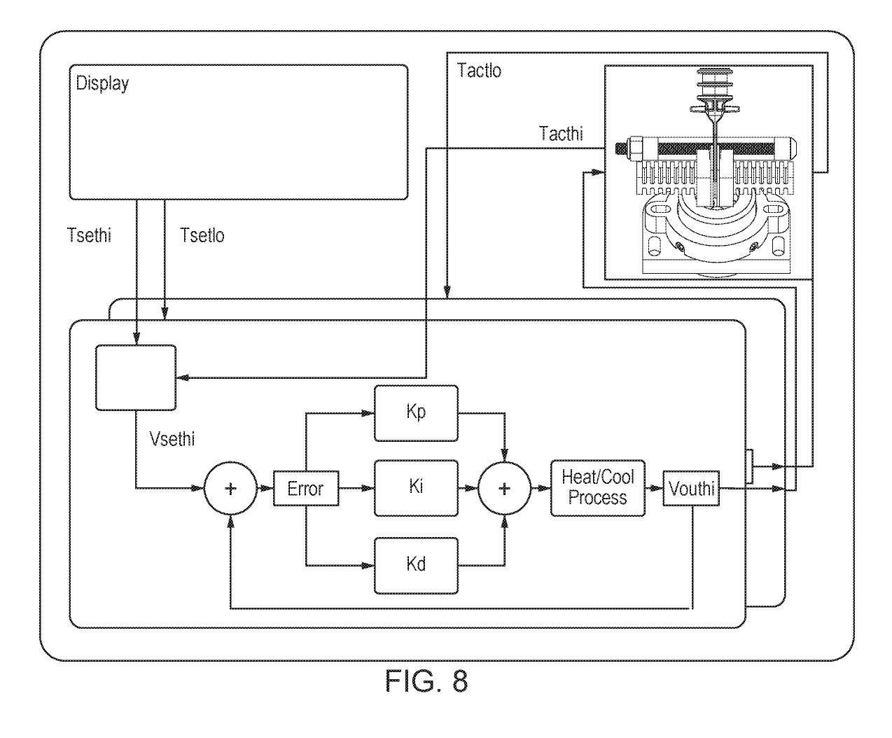

With reference to FIG. 8, in some embodiments, an example combined electronic/mechanical diagram illustrating the use of feedback-based proportional-integral-derivative (PID) controller to control temperatures of thermal transfer blocks is shown.

In some embodiments, the system of FIG. 8 may be enclosed within an outer box and may include a display configured to interact with a processor that can drive 2 ct or more controllers. The controllers and/or processors may be configured to implement control loop. In some embodiments, the controllers can drive the thermos-cycling device, and the temperature sensors feedback as inputs to the control loops.

Dynamic Flux Amplification

Generally, the present disclosure relates to nucleic acids as well as the devices, systems, and methods for using the same in conjunction with a method of DNA amplification hereinafter referred to as “Dynamic Flux Amplification” or “DFA.” In some embodiments, the term(s) “XCR”, “extreme chain reaction”, and/or variants thereof, can be interchangeably used with the terms “DFA”, “dynamic flux amplification”, and/or variants thereof

Generally, DFA refers to specific techniques of DNA and RNA amplification. DFA takes advantage of the fact that DNA amplification can take place within a fairly narrow temperature range. Once the Tm of the sequence of interest is determined, the DNA sample may be heated to that temperature or 1 to 5 degrees C. above that temperature. This defines the upper parameter of the heating and cooling cycle. The Tm of either the primers or the probes, (whichever possesses the lower Tm) defines the lower parameter of the heating and cooling cycle, within 1 to 5 degrees C.

In practicing DFA, it is generally preferred to use primers with a Tm as close as possible to the Tm of the sequence of interest so that the temperature may be cycled within a narrow range. The result of this narrow cycling is a dynamic opening and closing of a duplex between complementary nucleic acids comprising the sequence of interest as opposed to the complete, or nearly complete denaturing of the entire DNA strand.

The present existing primers (e.g., primers that were tested) target nucleic acid product that contains fewer nonspecific products. Thus, the amplified target nucleic acids products can be overall more specific and sensitive for quantitative PCR and genotyping target detection applications as described herein.

“Rational design” of oligonucleotide primers can include the selection via calculation, experiment, or computation of primers that have the desired melting temperature (Tm). The rational design can include selection of a specific primer sequences with the appropriate CG to obtain the desired Tm. Also, the rational design can include modifications to the primers that include internucleotide modifications, base modifications, and nucleotide modifications.

DFA Primer Design Methodology

In some embodiments, methods are provided for selecting primers for DFA that flank a variable sequence element of interest on a target nucleic acid.

In some embodiments, primers are selected to have a Tm with the target nucleic acid (primer:target Tin) that is within a narrow range of the Tm of the target nucleic acid (target:target Tm). The specific, narrow temperature range used for such an amplification of the target nucleic acids is dependent on the melting profile of the target nucleic acid, and thereby the sequence of the target nucleic acid being amplified. As such, the narrow temperature range can be used as a target temperature range in order to identify and/or generate specific primers that have sufficiently high Tm values when hybridized with the target nucleic acid.

DFA Primer Design—Overlapping Annealing/Denaturing Curves

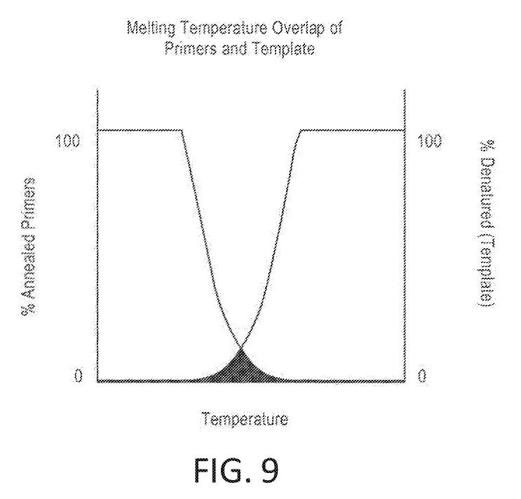

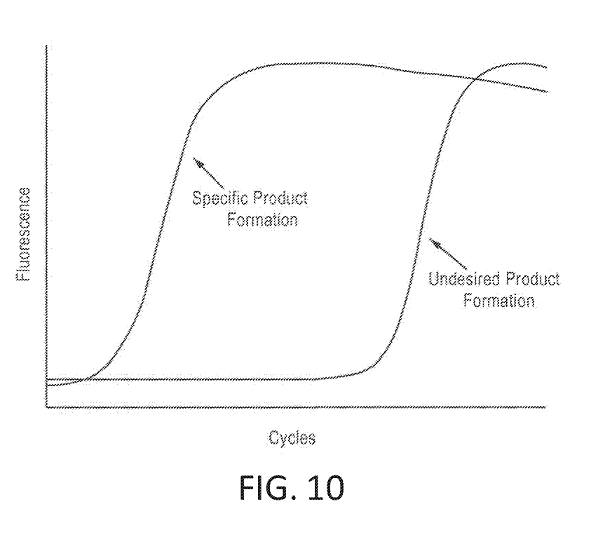

Accordingly, the Tm values of the primers can be overlapping within the temperature range of annealing and/or denaturing of the target nucleic acid (See, FIG. 9). FIG. 9 can be contrasted with FIG. 10 to illustrate the design of the primers to have the Tm within a range of the Tm of the target nucleic acid. FIG. 10 shows that conventional amplification with primers and a target nucleic acid are devoid of having a temperature overlap (as shown in FIG. 9) and require extreme temperature variations during amplification, corresponding to denaturation, annealing and extension cycles, to produce an amplified product. Such extreme temperature ranges allow for the formation of undesired products.

DFA Primer Design—Iterative Design

In some embodiments, an iterative design process is provided to select and/or optimize primers for specific target nucleic acid sequences to be amplified and/or detected. Advantageously, the iterative method enables the formation of a specific target nucleic acid by using a narrow range of thermal conditions where both the target nucleic acid and the oligonucleotide primers hybridized with the target nucleic acid are in a dynamic flux of annealing and denaturing. Such a dynamic flux of annealing and denaturing can result in a specific amplification of the target nucleic acid with a commensurate decrease in the formation of nonspecific amplification products. The implications of such iterative methods for selecting and/or optimizing primers provides for the use of low cost dyes in lieu of more expensive custom oligonucleotide probes, such as those having fluorescent labels, can allow for quantitative PCR or high resolution denaturation to be used in analyzing the sequence of the target nucleic acid. Also, the iterative method can provide primers that function in the absence of exquisite thermally controlled instruments for the formation of amplification products.

That is, the primers can operate within a narrow temperature range in order to amplify the target nucleic acid, allowing nucleic acid amplification to be used in a much broader range of uses. A number of methods have been described in the art for calculating the theoretical Tm of DNA of known sequence, including, e.g., methods described by Rychlik and Rhoads, Nucleic Acids Res. 17:8543-8551 (1989); Sambrook, J. et al., Molecular Cloning: A Laboratory Manual, Cold Spring Harbor Laboratory Press, Cold Spring Harbor, N.Y. (1989); and Breslauer et al., Proc Natl Acad. Sci. 83: 3746-3750 (1986).

Such an iterative process can include identifying an initial target nucleic acid sequence as the target amplicon, wherein the target nucleic acid sequence can be associated with a particular biological activity, such as possible drug resistance. The target nucleic acid sequence is then amplified in order to produce an amplified product, and the Tm value of the amplified product (e.g., amplicon) is determined using conventional melting curve analysis. The melting curve analysis is then utilized to determine or compute new primers or primer sets for use in the amplification of the target nucleic acid.

The determined or computed primers are then designed with primer Tm values within the range of the melting peak generated by the melt of the amplified product. The primers are then prepared or synthesized to have the designed primer Tm values.

DFA Primer Design—Oligonucleotide Chemical Modification

In some embodiments, primers can be configured to have a Tm that is within a narrow range of the Tm of the target nucleic acid by chemically modifying the oligonucleotides. Well known oligonucleotide synthesis chemistries may be used to increase the Tm values of the primers so they correspond to the temperature range of the Tin of the target nucleic acid. Such chemistries may use modified bases (e.g., Super G, A, T, C), LNA, or PNA, or other such oligonucleotide stabilizing chemistries. Also, additional oligonucleotide hybridization stabilizing chemistries may be developed that can be used for this application.

For example, primers synthesized with both conventional phosphodiester linkage chemistry, and LNA chemistries have been used to provide primer Tm values close to the Tm values of the target nucleic acid sequence. However, it is possible that certain target nucleic acids may have Tm values lower than that of the primers, and a hybridization destabilizing chemistry may need to be included to decrease the primer Tm values so that the primer Tm value is within a range of the Tm values of the target nucleic acid sequence.

DFA Primer Design—Melting Curve Analysis

In some embodiments, methods are provided for refining the design of the primers to minimize the temperature range for the specific amplification of the target nucleic acid sequence. As such, the target nucleic acid is amplified with standard reaction thermal cycling conditions to ensure the target nucleic acid sequence is amplified. The amplification is monitored using real-time PCR with a double-stranded DNA binding dye, such as SYBR, LCGreen, LCGreen+, Eva dye, or the like.

The amplified target nucleic acid is subjected to a melting curve analysis to determine the actual Tm value of the target nucleic acid sequence. The melting peak, which can be expressed as -HAT, is generated from melting the amplified target nucleic acid and can have a range similar to a distribution curve across a defined temperature range. At the low temperature end, the amplified target nucleic acid template is partially denatured. At the uppermost temperature the entire sample of amplified target nucleic acid is denatured. The temperature necessary to denature the target nucleic acid during the amplification procedure is within this temperature distribution.

Initially, the uppermost temperature is recommended to ensure more complete denaturation. Subsequently, the lowermost temperature of the distribution curve can be used as the initial Tm for a set of designed primers for use in amplification before any iterative changes are made to the primers.

Confirmation of the narrow temperature range that the initial primers may be used with can be performed either in serial or in parallel experiments of ever increasing annealing temperatures.

Alternatively, the individual primers can be added to the amplified template and an additional melting curve analysis can be performed on the combined primer and template melting curves.

In any event, the Tm of the primers can be configured to overlap with a narrow temperature range that contains the Tm of the target nucleic acid sequence. The highest annealing temperature from these experiments where the target nucleic acid sequence is amplified specifically and efficiently can be considered the temperature which defines the optimal annealing temperature for the existing primers (e.g. primers that were tested). These same primers or slightly modified primers can then be resynthesized with additional hybridization stabilizing chemistries. Modifications to the primers to change the Tm in the desired direction so that the primer Tm overlaps with a narrow temperature range that contains the Tm of the target nucleic acid sequence. This can be accomplished using online design tools, such as the LNA design tool available from Integrated DNA Technologies. Such design tools can be used to estimate the number of necessary LNA, modifications required to raise the Tm of the primer to better overlap with the melting curve of the target nucleic acid sequence.

In the instance the primer Tm values are greater than the highest melting temperature of the target nucleic acid sequence, it may be necessary to redesign the primers to have a lower Tm. Alternatively, the quantity of divalent and/or monovalent cation salts or other destablizing agents (e.g., AgCl, DMSO, etc.) that are used in the amplification protocol (e.g., PCR) may be reduced to destabilize the hybridization of these oligonucleotides to the template. In any event, a reduction in the primer Tm may be needed in some instances.

DFA Primer Design—GC Content Modification

In some embodiments, the primer Tm can be modified by altering the GC content of the primer sequence. By changing the GC content, the primer Tm can be selectively changed. Usually, increasing the GC content can increase the Tm, and decreasing the GC content can decrease the Tm. However, there are instances that a high GC content is desired that will overly increase the Tm. In such instances, destabilizers can be used to enable the inclusion of high GC content primers or for the use of high GC content target nucleic acid sequences. The de-stabilizers can selectively decrease the temperature of the amplification procedure. Examples of destabilizers include DMSO, AgCl, and others.

DFA Thermal Cycling Ranges

In some embodiments, the primers can be prepared so that the target nucleic acid amplification or enrichment protocols can be performed at minimized temperature differences during the thermal cycling. This allows the thermal cycling to be done within a narrow temperature range so as to promote the formation of a specific product.

One range of thermal cycling can be within about 15° C. of the target nucleic acid Tm, or within 10° C. of the target nucleic acid Tm, or within 5° C. of the target nucleic acid Tm, or within 2.5° C. of the target nucleic acid Tm, or within 1° C. of the target nucleic acid Tm or even substantially the same Tm as that of the target nucleic acid Tm.

In some embodiments, the thermal cycling conditions for the amplification of the target nucleic acid spans the range of the Tm peak +/− about 1° C. to 15° C. of the target nucleic acid sequence

In some embodiments, the thermal cycling conditions for the amplification of the target nucleic acid spans the range of the Tm peak +/− about 1° C. to 10° C. of the target nucleic acid sequence.

Or, in some embodiments, the thermal cycling conditions for the amplification of the target nucleic acid spans the range of the Tm peak +/− about 1° C. to 5° C. of the target nucleic acid sequence.

In some embodiments, the thermal cycling conditions for the amplification of the target nucleic acid spans the range of the Tm peak +/− about 5° C. to 15° C. of the target nucleic acid sequence.

In some embodiments, the thermal cycling conditions for the amplification of the target nucleic acid spans the range of the Tm peak +/− about 5° C. to 10° C. of the target nucleic acid sequence.

In some embodiments, the thermal cycling conditions for the amplification of the target nucleic acid spans the range of the Tm peak +/− about 5° C. of the target nucleic acid sequence.

In some embodiments, the thermal cycling conditions for the amplification of the target nucleic acid spans the range of the Tm peak +/− about 2.5° C. of the target nucleic acid sequence.

Such narrow temperature ranges make it possible to amplify specific target nucleic acids without thermal cycling between temperatures corresponding to the normal stages of PCR amplification (denaturation, annealing, and extension).

Also, it makes it possible to perform amplifications and enrichments in commercial temperature-controlled instruments that can be set at selected temperatures or be varied within narrow temperature ranges, such as an oven, heating block, or the like.

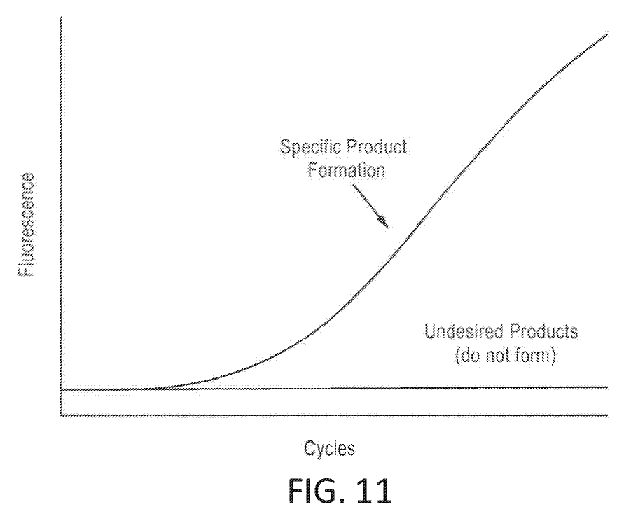

FIG. 11 illustrates the graph of a narrow temperature range PCR amplification with the same target nucleic acid sequence as shown in FIG. 10, which shows more specific product formation and less undesired products are formed.

In some embodiments, the temperatures of the thermocycling can be selected in a narrow temperature range to substantially limit amplification to amplifying the target nucleic acid sequence. As such, the thermal cycling conditions can be modified to amplify the target nucleic acid sequence by modifying the annealing temperature to be substantially the same as the lower temperature base of the melting peak for the amplicon. Also, the thermal cycling conditions can be modified to amplify the target nucleic acid sequence by modifying the annealing temperature to be substantially the same as the higher temperature base for the melting peak of the amplicon.

In some embodiments, the primer Tm can be selected so that the amplification of the target nucleic acid can be performed at a temperature that ranges between about 75° C. to about 90° C. Such a temperature range, or narrowed 5° C. to 10° C. range therein, can be used for the amplification of DNA and/or RNA target nucleic acid sequences to reduce the formation of non-specific products during the amplification (e.g., PCR) process.

In some embodiments, the primer Tin can be selected so that the amplification is performed at isothermal amplification conditions in the Tm range of the target nucleic acid sequence to ensure appropriate product formation.

In some embodiments, the present disclosure includes a method of designing a primer set having a Tm with a target nucleic acid that is within a narrow range from the Tm of the target nucleic acid sequence. As such, the primer set can be designed so that the primer Tm overlaps the distribution curve of the Tm of the target nucleic acid sequence. For example, the primer set can be used in real-time PCR assays so that the primer Tm overlaps the distribution curve of the Tm for the target nucleic acid sequence so that a narrow temperature range can be used to amplify the target nucleic acid sequence.

DFA pH Modification

In some embodiments, the conditions of the protocol for amplifying the target nucleic acid sequence can be modified to an appropriate pH to increase the specificity of selectively amplifying the target nucleic acid over other nucleic acids. As such, the use of an appropriate pH can increase the ability to selectively amplify the target nucleic acid sequence. This can include the use of an amplification buffer that can enable the activation of chemically inactivated thermal stable DNA polymerases. Also, adjusting the pH with selected amplification buffers can allow for the amplification protocol to be performed at reduced temperatures, such as those temperatures ranges that have been recited herein.

In some embodiments, the pH of the amplification buffer can be adjusted so as to allow for the conversion of a chemically inactivated enzyme to the activated state. As such, an enzyme may be activated in a slightly acidic condition; however, basic pH values may be used for some enzymes. For acid-activated enzymes, standard Tris-based PCR buffers can have significant temperature dependence (e.g., reducing by 0.028 pH units per degree C.). Complete activation of the enzyme (e.g., chemically inactivated thermal stable DNA polymerase) from the inactivated state can require the pH to be less than about 7, more preferably less than about 6.75, and most preferably less than 6.5.

In some embodiments, the amplification protocol includes the use of lower pH buffers so that the amplification can be performed at lower activation temperatures. For example, for every 10° C. below 95° C., the enzyme activation temperature can be lowered by 0.3 pH units. However, limits to this approach are entirely a function of the dye chemistry used for the real-time detection of the amplified template (e.g., Fluorescein-based detection has significantly reduced fluorescence below pH 7.3).

DFA Modulation of Ainplicon Size

In some embodiments, the design of the primers and/or amplification conditions can be modulated so as to modulate the size of the target nucleic acid sequence being amplified. This can include modulating the design of the primers and/or amplification conditions so that the size of the amplicon is significantly larger than that of the combined primers only. This can include the amplicon being 1-3 nucleotides longer than the primers, or 2 times larger than the primers, or 5 times larger than the primers, and more preferably 10 times larger than the primers.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be an example and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

At least some of the embodiments disclosed above, in particular at least some of the methods/processes disclosed, may be realized in circuitry, computer hardware, firmware, software, and combinations thereof (e.g., a computer system). Such computing systems, may include PCs (which may include one or more peripherals well known in the art), smartphones, specifically designed medical apparatuses/devices and/or other mobile/portable apparatuses/devices. In some embodiments, the computer systems are configured to include clients and servers. A client and server are generally remote from each other and typically interact through a communication network (e.g., VPN, Internet). The relationship of client and server arises by virtue of computer programs running on the respective computers and having a client-server relationship to each other.

One or more embodiments described herein relate to a computer storage product with a non-transitory computer-readable medium (also can be referred to as a non-transitory processor-readable medium) having instructions or computer code thereon for performing various computer-implemented operations. The computer-readable medium (or processor readable medium) is non-transitory in the sense that it does not include transitory propagating signals per se (e.g., a propagating electromagnetic wave carrying information on a transmission medium such as space or a cable). The media and computer code (also can be referred to as code or algorithm) may be those designed and constructed for the specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code disclosed herein.

One or more embodiments and/or methods described herein can be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) can be expressed in a variety of software languages (e.g., computer code), including C, C++, Java®, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

Some embodiments of the disclosure (e.g., methods and processes disclosed above) may be embodied in a computer program(s)/instructions executable and/or interpretable on a processor, which may be coupled to other devices (e.g., input devices, and output devices/display) which communicate via wireless or wired connect (for example).

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms.

As noted elsewhere, the disclosed embodiments have been presented for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with claims supported by the present disclosure and their equivalents. Moreover, embodiments of the subject disclosure may include methods, compositions, systems and apparatuses/devices which may further include any and all elements from any other disclosed methods, compositions, systems, and devices, including any and all elements corresponding to detecting one or more target molecules (e.g., DNA, proteins, and/or components thereof). In other words, elements from one or another disclosed embodiments may be interchangeable with elements from other disclosed embodiments. Moreover, some further embodiments may be realized by combining one and/or another feature disclosed herein with methods, compositions, systems and devices, and one or more features thereof, disclosed in materials incorporated by reference.

In addition, one or more features/elements of disclosed embodiments may be removed and still result in patentable subject matter (and thus, resulting in yet more embodiments of the subject disclosure). Furthermore, some embodiments correspond to methods, compositions, systems, and devices which specifically lack one and/or another element, structure, and/or steps (as applicable), as compared to teachings of the prior art, and therefore represent patentable subject matter and are distinguishable therefrom (i.e. claims directed to such embodiments may contain negative limitations to note the lack of one or more features prior art teachings).

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An apparatus, comprising:
a reaction vessel including a first wall and an opposing second wall positioned so as to define a sample chamber therebetween, a width of the sample chamber being less than about 2 mm; and
a heat source configured to vary a first temperature of the first wall and a second temperature of the second wall such that a temperature difference between the first temperature and the second temperature induces thermal cycling in a solution contained within the sample chamber of the reaction vessel.

2. The apparatus of claim 1, wherein the maximum width of the sample chamber is in a range from about 0.5 mm to about 1.0 mm.

3. The apparatus of claim 1, wherein the reaction vessel is a closed system.

4. The apparatus of claim 1, wherein the heat source includes a first thermal transfer block in thermal communication with the first wall and a second thermal transfer block in thermal communication with the second wall.

5. The apparatus of claim 1, wherein the heat source includes a first plate in direct thermal contact with a side of the first wall, and a second plate in direct thermal contact with a side of the second wall.

6. The apparatus of claim 1, wherein the heat source includes a first Peltier device for varying the temperature of the first wall and a second Peltier device for varying the temperature of the second wall.

7. The apparatus of claim 1, wherein the temperature difference is in a range from about 10° C. to about 40° C.

8. The apparatus of claim 1, wherein the temperature difference is in a range from about 40° C. to about 75° C.

9. A system, comprising:
an apparatus including:
a reaction vessel including a first wall and an opposing second wall positioned so as to define a sample chamber therebetween, a maximum width of the sample chamber being less than about 2 mm; and
a heat source including a first thermal transfer block and/or a second thermal transfer block, the first thermal transfer block in thermal communication with the first wall and configured to vary a first temperature of the first wall, the second thermal transfer block in thermal communication with the second wall and configured to vary a second temperature of the second wall;
a processor, operatively coupled to the heat source, and configured to determine a characteristic of a current for flowing through: the first thermal transfer block so as to establish the first temperature of the first wall, and/or the second thermal transfer block so as to establish the second temperature of the second wall;
a light source configured for irradiating the solution in the sample chamber of the reaction vessel; and
a detector, operatively coupled to the reaction vessel, the detector configured for detecting fluorescence emitted by the solution in the sample chamber.

10. The system of claim 9, wherein the maximum width of the sample chamber ranges from about 0.5 mm to about 1.0 mm.

11. The system of claim 9, further comprising:
a conduit for transmitting the fluorescence light exiting the sample chamber to the detector.

12. The system of claim 9, wherein the characteristic of the current includes an amount and/or direction of the current flowing through the first thermal transfer block and/or the second thermal transfer block.

13. The system of claim 9, wherein the characteristic of the current includes a duration of time for flowing the current through the first thermal transfer block and/or the second thermal transfer block.

14. The system of claim 9, wherein a difference of the first temperature and the second temperature ranges from about 0° C. to about 90° C.

15. The system of claim 9, further comprising:
a display, operatively coupled to the detector, and configured to display a graphical representation of the fluorescence from the sample chamber.

16. The system of claim 9, wherein the detector includes a smartphone.

17. A method, comprising:
forming a reaction vessel including a first wall and an opposing second wall positioned so as to define a sample chamber therebetween, a width of the sample chamber being less than about 2 mm; and
coupling a heat source to the first wall and the second wall, the heat source configured to vary a first temperature of the first wall and a second temperature of the second wall such that during use, a temperature difference between the first temperature and the second temperature induces thermal cycling in a solution contained within the sample chamber of the reaction vessel.

18. A method, comprising:
establishing, via a heat source, a temperature difference between a first wall and an opposing second wall of a reaction vessel, the first wall and the second wall arranged in close proximity to each other so as to define a sample chamber therebetween, the sample chamber having a width of less than about 2 mm;
irradiating the sample chamber with light, the sample chamber having a solution disposed therein; and
detecting, via a detector operatively coupled to the reaction vessel, fluorescence emitted by the solution,
wherein the temperature difference between the first wall and the second wall is configured to induce thermal cycling of the solution in the sample chamber.

19. The method of claim 18, wherein establishing a temperature difference between the first wall and the second wall includes varying a first temperature of the first wall and/or a second temperature of the second wall by flowing a current through a first thermal transfer block and/or a second thermal transfer block, the first thermal transfer block and/or the second thermal transfer block being in thermal communication with the first wall and/or the second wall, respectively.

20. The method of claim 19, further comprising:
determining, via a processor operatively coupled to the first thermal transfer block and/or the second thermal transfer block, an amount, a direction and/or a duration of the current to flow through the first thermal transfer block and/or the second thermal transfer block to maintain the first temperature and/or the second temperature, respectively.

21. The method of claim 18, further comprising:
displaying, at a display of the detector and/or an external display operatively coupled to the detector, a graphical representation of the fluorescence light emitted by the content of the sample chamber.
